(19) United States
(12) Patent Application Publication
(10) Pub. No.: US 2022/0087828 A1
(43) Pub. Date: Mar. 24, 2022

(54) PARTIAL GLENOID ARTHROPLASTY

(71) Applicant: TIGON MEDICAL, Millersville, MD (US)

(72) Inventors: Umasuthan SRIKUMARAN, Ellicott City, MD (US); Colby GEARY, Baltimore, MD (US); Jeremy CLARK, Millersville, MD (US); Mohit GILOTRA, Baltimore, MD (US); Stephan PILL, Greenville, SC (US); Surena NAMDARI, Gladwyne, PA (US); Peter Johnston, Leonardtown, MD (US); Kelly KILCOYNE, Bethesda, MD (US); Gabriel HORNEFF, Garnet Valley, PA (US)

(21) Appl. No.: 17/479,947

(22) Filed: Sep. 20, 2021

Related U.S. Application Data
(60) Provisional application No. 63/080,747, filed on Sep. 20, 2020.
(60) Provisional application No. 63/182,629, filed on Apr. 30, 2021.

(51) Int. Cl. A61F 2/40; A61F 2/28; A61F 2/30; A61F 2/46

(52) U.S. Cl. CPC A61F 2/4081; A61F 2/2846; A61F 2/30749; A61F 2/4612; A61B 17/1684; A61F 2002/30892; A61F 2310/00359; A61F 2310/00796; A61F 2002/285

(57) ABSTRACT

Disclosed are devices, methods and/or systems for a partial glenoid implant for use during glenoid arthroplasty, including related surgical methods and instruments.

### CROSS-REFERENCE TO RELATED APPLICATIONS

This application claims priority to and benefit thereof from U.S. Provisional Patent Application No. 63/080,747 filed Sep. 20, 2020, titled “PARTIAL GLENOID ARTHROPLASTY,” and U.S. Provisional Patent Application No. 63/182,629 filed Apr. 30, 2021, titled “PARTIAL GLENOID ARTHROPLASTY,” the disclosures of which are each incorporated by reference herein in their entireties.

### TECHNICAL FIELD

The invention relates to improved devices, systems and methods for a partial glenoid implant for use during glenoid arthroplasty, as well as related surgical methods and instrument kits.

### BACKGROUND OF THE INVENTION

Shoulder instability or Glenohumeral Instability is a common problem in a large proportion of the population. In many cases, shoulder instability associated with glenoid (i.e., the shoulder socket) bone loss is more likely to result in recurrent instability compared to soft tissue injuries (i.e., to the capsule or labral) alone. Moreover, recurrent or repeated instability, even after surgical procedures, can result in significant morbidity to the shoulder, including (1) limited function, (2) ongoing dislocation events, and/or (3) premature onset of arthritis and the need for early joint replacements later in life.

In addition to capsular plication and/or labral repair, there are many options that exit that seek to restore glenoid bone stock, including autograft implantation (i.e., from the iliac crest and/or distal clavicle), coracoid bone and/or muscle transfer (i.e., Latarjet/Bristow), and/or allograft implants (i.e., for a distal tibia). However, each approach has various advantages and concomitant disadvantages ad significant limitations.

Typically, a partial shoulder prosthesis would include a glenoid implant intended to replace some portion of the glenoid cavity of the scapula (which may be utilized in combination with a humeral implant intended to replace the humeral head). The glenoid implant may generally includes an articular portion intended to articulate with and/or stabilize the humeral head within the joint, and may include various types of fixation to stabilize the articular body with respect to the scapula.

### SUMMARY OF THE INVENTION

The various inventions disclosed herein include the realization of a need for improved methods, apparatus and/or systems for glenoid arthroplasty. In various embodiments, the solution of a partial glenoid arthroplasty is proposed, where a portion of the glenoid can be augmented using a kit of partial glenoid implants of differing shapes and/or sizes, which may be utilized with allograft and/or autograft tissue grafts to desirably secure and/or stabilize the tissue grafts on the glenoid, thereby reducing surgical time, reducing patient pain, speeding patient recovery and/or improving function of the joint after surgery.

In some alternative embodiments, the various implant components may be utilized to replace glenoid bone loss without need for harvesting a graft and/or fashioning a bone graft of appropriate shape and/or size. In various embodiment, the implants can comprise a metallic material (i.e., titanium, cobalt chrome, or any other suitable biocompatible implant material), and in various embodiments the implant can include a textured or grit surface, such as a hydroxyapatite grit surface, to promote bone ingrowth. The use of such materials may be appropriate as they are highly similar to currently accepted arthroplasty materials with a long history of biocompatibility, they provide great initial and long-term strength without a risk of resorption over time, and they have a high likelihood of causing bony ingrowth (i.e., similar to the long history of bone ingrowth for hydroxyapatite grit surfaces in implants of many other surgical applications.)

In at least one exemplary embodiment, a kit of partial glenoid implants of differing sizes and/or shapes can be provided, with shapes and/or curvatures that desirably match or substantially match native glenoid shapes (either general anatomical shapes and/or shapes manufactured and/or selected to match the natural anatomy of an intended patient). Desirably, such a kit will reduce and/or obviate the need for fashioning an appropriately shaped and/or sized implant during a surgical procedure (thereby shortening surgery and operating room times). For example, an exemplary kit could comprise a set of nine different size options of glenoid implants in a single surgical tray, wherein the implants could be designed for symmetrical usage (i.e., appropriate for left or right shoulder implantation). Various kit embodiments could also optionally include surgical tools and/or insertion equipment to ensure appropriate positioning of the implant relative to the native and/or modified articulating surface.

### BRIEF DESCRIPTION OF THE SEVERAL VIEWS OF THE DRAWINGS

The foregoing and other objects, aspects, features, and advantages of embodiments will become more apparent and may be better understood by referring to the following description, taken in conjunction with the accompanying drawings, in which:

FIG. 1 depicts an anterio-lateral view of a scapula, which includes a glenoid cavity;

FIG. 2A depicts a perspective view of one exemplary embodiment of a glenoid plate for use in repairing a damaged glenoid;

FIG. 2B depicts another perspective view of the glenoid plate of FIG. 2A;

FIG. 3 depicts a phantom view of various internal structures of the glenoid plate of FIGS. 2A and 2B;

FIG. 4 depicts various dimensions of an exemplary glenoid plate that may be altered in a kit containing multiple implants of varying shapes and/or sizes;

FIG. 5 depicts a bottom plan view of the glenoid plate of FIG. 3,

FIGS. 6A through 6C depict various views of a fixation bolt for use with a glenoid plate;

FIGS. 7A through 7E depict various views of one exemplary embodiment of a graft that can be utilized with the glenoid plate of FIG. 3;

FIG. 8A depicts an exemplary placement of a graft and the glenoid plate of FIG. 3 on a glenoid of a patient;

FIG. 8B depicts a perspective phantom view of various internal structures of the graft and the glenoid plate of FIG. 8A;

FIG. 8C depicts another perspective phantom view of various internal structures of the graft and the glenoid plate of FIG. 8A;

FIG. 9 depicts a close-up partial view of various internal structures of the glenoid plate, graft, guide pins and fixation bolt of FIG. 8B;

FIG. 10 depicts a perspective phantom view of a fully implanted graft and the glenoid plate;

FIG. 11 depicts an alternative placement technique for securing a graft and glenoid plate to a glenoid body;

FIG. 12 depicts a suture being drawn to pull a graft and glenoid plate into intimate contact with a grafting surface of a glenoid body;

FIG. 13 depicts sutures being advanced through a cannulated fixation bolt and a cannulated driving tool;

FIG. 14 depicts a phantom view of the graft and glenoid plate secured to the glenoid body, prior to suture removal;

FIG. 15A through 15D depict various views of another exemplary embodiment of a glenoid plate;

FIG. 15E depicts a cross sectional view of the glenoid plate of FIGS. 15A through 15D, implanted within a glenoid body;

FIG. 16 depicts one exemplary surgical kit for partial glenoid repair; and

FIG. 17 depicts another alternative embodiment of a glenoid plate and surgical placement technique.

### DETAILED DESCRIPTION OF THE INVENTION

The disclosures of the various embodiments described herein are provided with sufficient specificity to meet statutory requirements, but these descriptions are not necessarily intended to limit the scope of the claims. The claimed subject matter may be embodied in a wide variety of other ways, may include different steps or elements, and may be used in conjunction with other technologies, including past, present and/or future developments. The descriptions provided herein should not be interpreted as implying any particular order or arrangement among or between various steps or elements except when the order of individual steps or arrangement of elements is explicitly described.

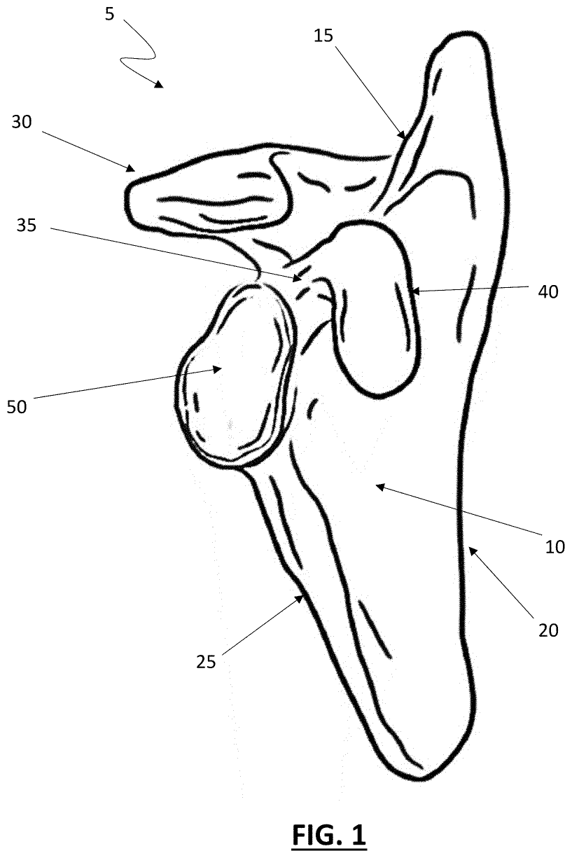

FIG. 1 depicts an anterio-lateral view of a scapula 5, which includes a scapular body 10, a superior border 15, a medial border 20, a lateral border 25, an acromion process 30, a spine 35, a coracoid process 40 and a glenoid cavity 50. In many instances, severe glenoid bone loss (often to the anterior glenoid lip) can result in a socket deficiency which may necessitate structural bone-grafting to address the bone loss and build up the socket so that it provides more stability for the joint.

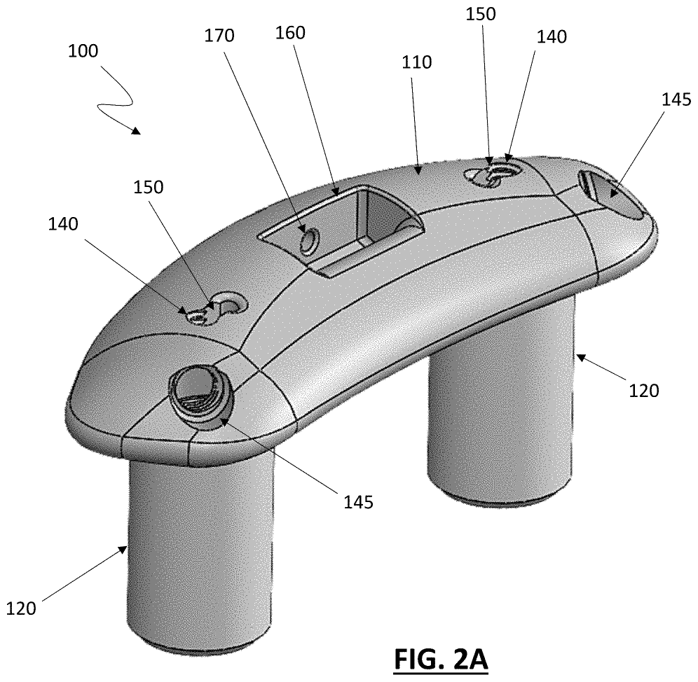

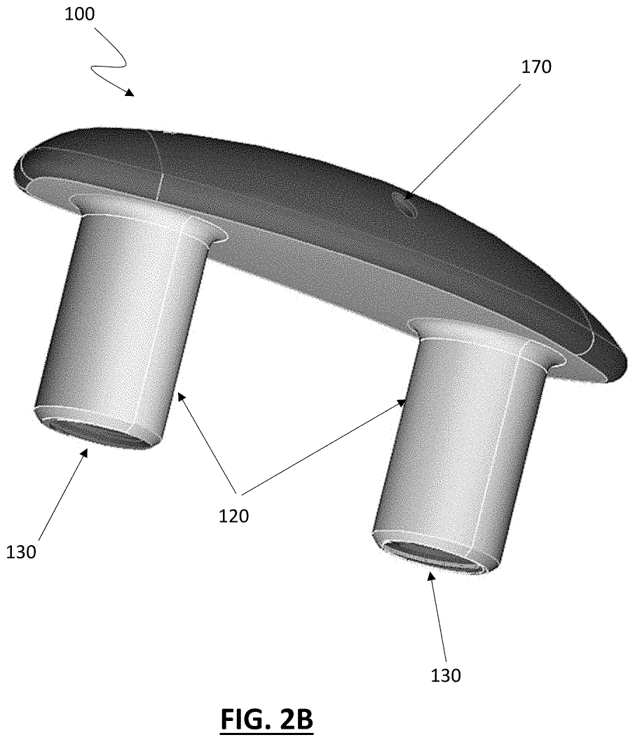

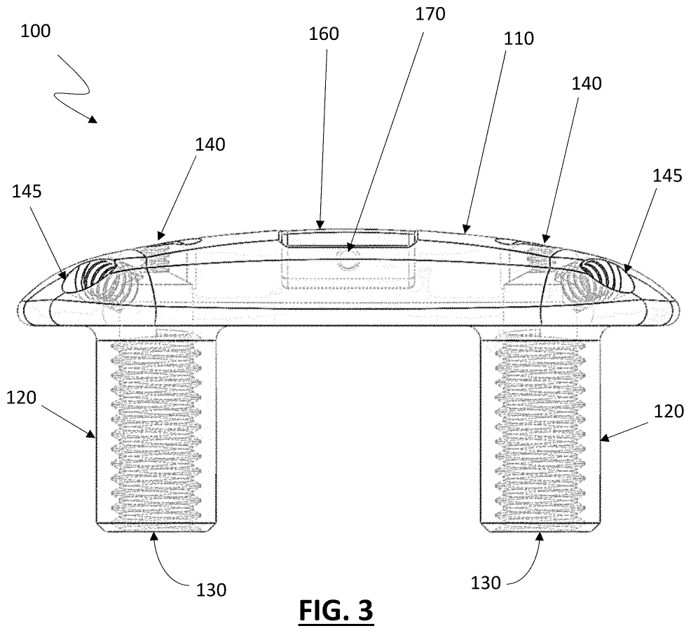

FIGS. 2A and 2B depict perspective views of one exemplary embodiment of a glenoid plate 100 for use in securing a graft to a damaged glenoid, with FIG. 3 showing various internal structures in phantom. The glenoid plate 100 include an elongated plate body 110 with a pair of legs 120 extending downward from the body 110, with each of the legs 120 including a longitudinally extending interior tubular leg opening 130 extending through the leg (see FIG. 3), with upper openings 140 extending through the body 110 (i.e., proximate to each leg opening 130). The body 110 also includes a pair of inserter holes 145 which can optionally be internally threaded (for engagement with an insertion tool, for example), with each inserter hole extending through the body into the tubular leg openings 130. A pair of slots 150 are formed in the body proximate to each of the upper openings 140. The body 110 also includes a slot 160 on an upper surface of the body to engage with and/or accommodate a drill guide or similar tool, with a hole 170 formed on the top of the plate to accommodate a suture (not shown) therethrough, which suture can be utilized as a retrieval device to remove the plate during possible retrieval surgery and/or could optionally be used to “tie down” soft tissue.

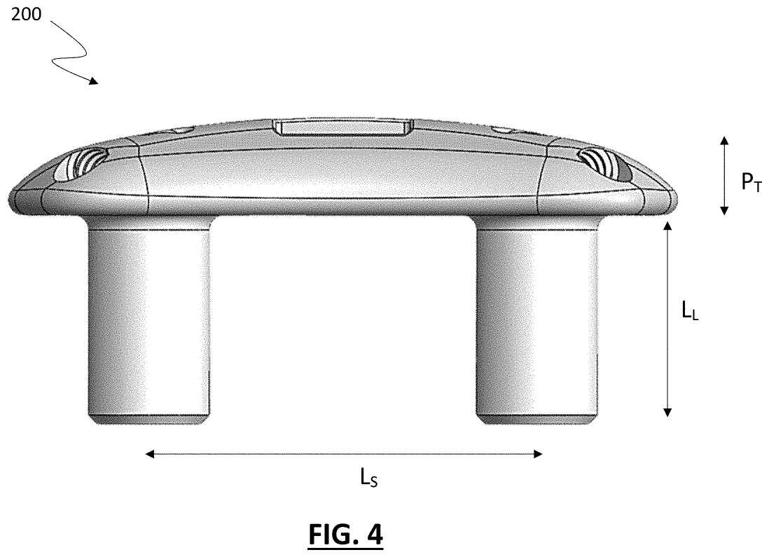

FIG. 4 depicts a side view of an exemplary glenoid plate 200, wherein various dimensional features of the glenoid plate 200 can be altered in a kit containing multiple implants of varying shapes and/or sizes. For example, a plate thickness P_{T} could be varied among implants in a kit, or a leg spacing L_{S} could be varied for different size implants. Similarly, a leg length L_{L} could be altered for different implants (which may optionally require altering a length of fixation screws). In at least one exemplary embodiment of a kit, a series of plates having differing plate thicknesses, with a set Leg Spacing L_{S} of approximately 13 mm apart (i.e., leg center to leg center) for all plates, could be utilized in various procedures.

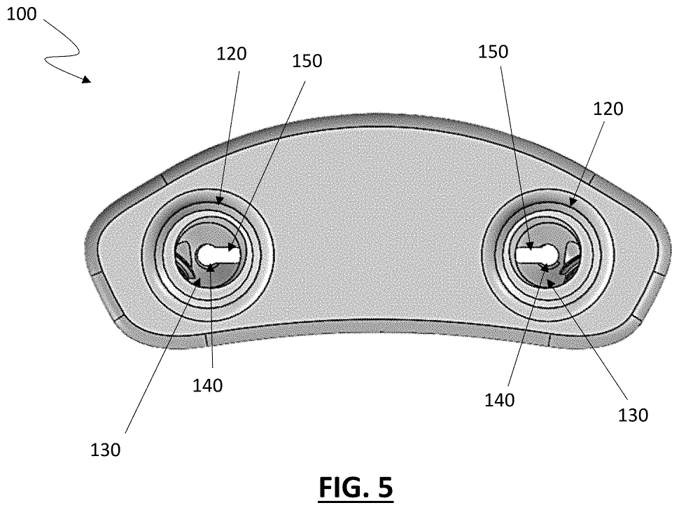

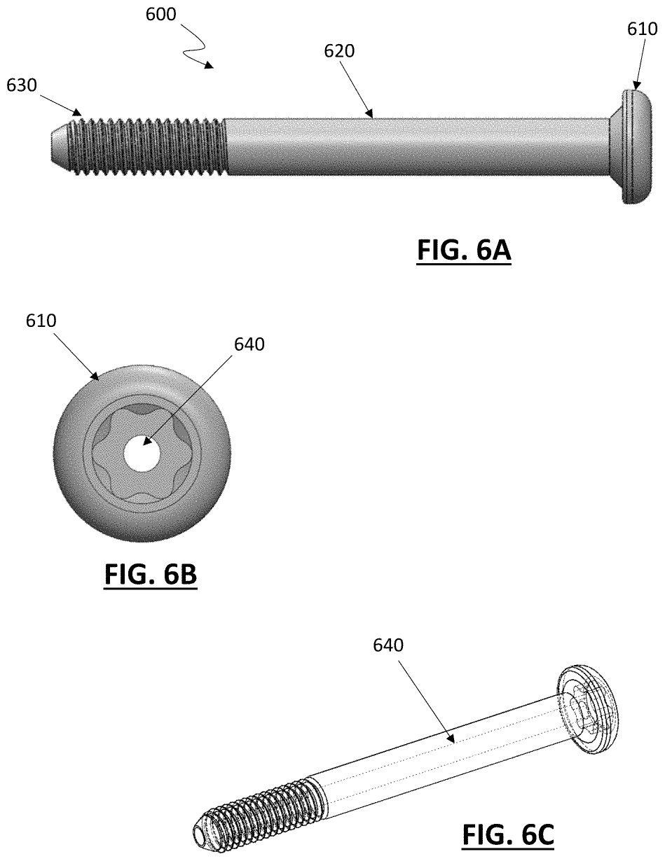

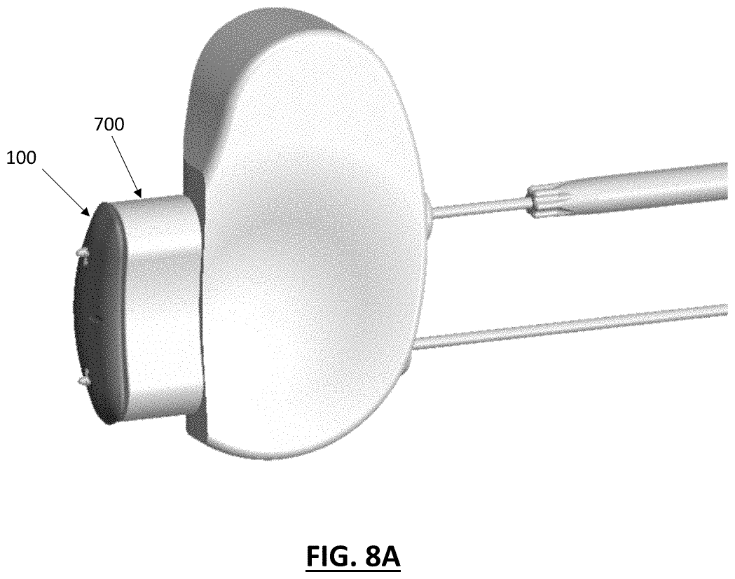

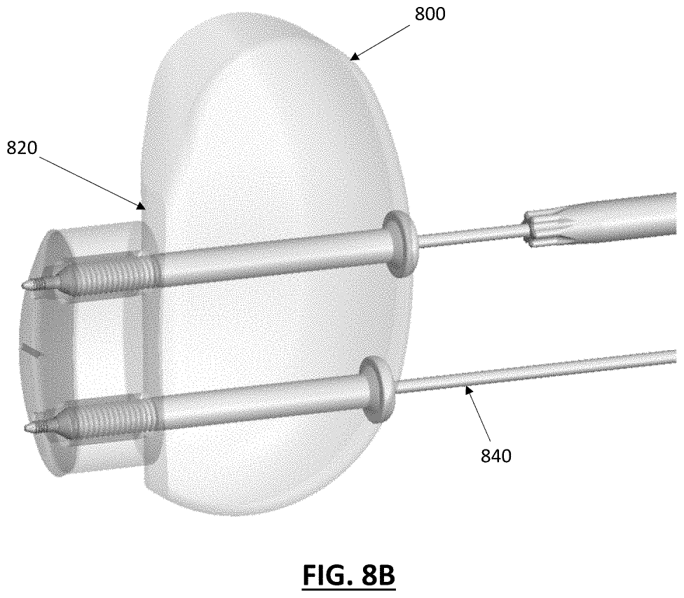

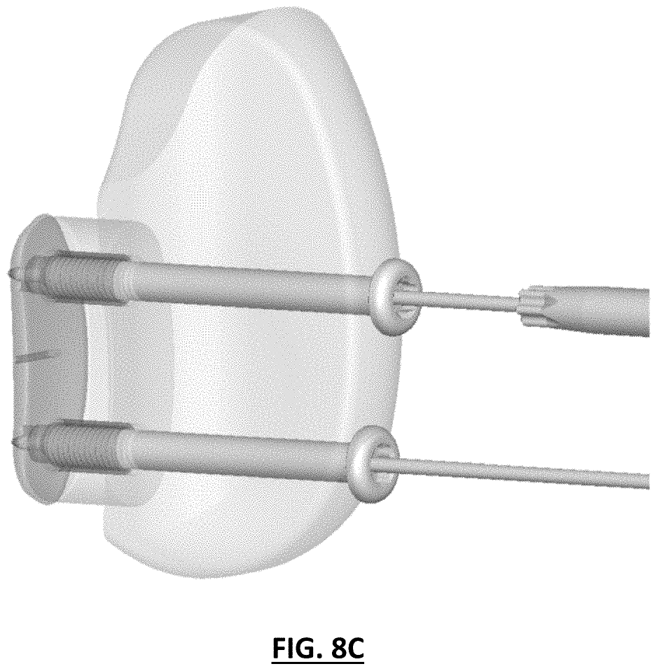

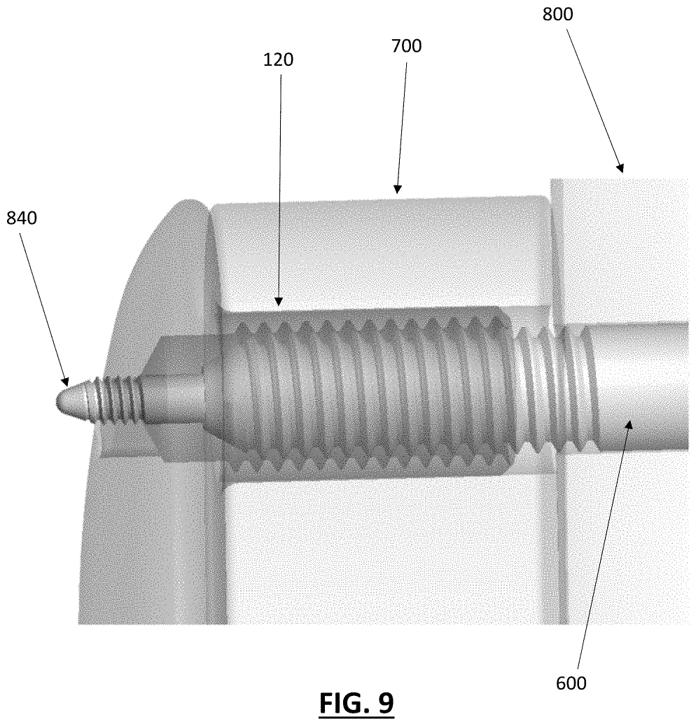

FIG. 5 depicts a bottom plan view of the glenoid plate 100 of FIG. 3, showing the cannulated tubular leg openings 130 extending through the legs 120, as well as the upper openings 140 and slots 150. In this embodiment, the tubular leg openings are internally threaded to accommodate a fixation bolt or pin (see FIG. 6A), and the upper openings 140 can also be internally threaded to accommodate the distal end of a threaded guide pin 840 (See FIGS. 8B and 9). Desirably, the slots 150 next to the upper openings 140 are positioned to allow suture to be fixed to the plate, allowing the physician to pull the plate down to the glenoid as desired.

The use of threaded guide wires in various embodiments allows the guide wires to be threaded into the plate, which allows for posterior control of the plate/graft, and also allows for guiding of the bolts during insertion. The guide wires ensure that the bolts are centered in the plate legs and provide for removability in the event that cross-threading may occur. Moreover the various threaded openings in the plate are oriented such that the bolts and the guide wires can be secured to the plate simultaneously, while also allowing for removal of the guide wires after the bolts have been inserted and/or optionally tightened.

FIGS. 6A through 6C depict various views of a fixation bolt 600 for use with a glenoid plate. The fixation bolt 600 can include a wide proximal head 610 which desirably will rest on a posterior surface of the glenoid when implanted, a central body section 620 which may be smooth, threaded and/or coated at the surgeon's option, and a threaded distal tip section 630 which is desirably sized to engage with a corresponding internally threaded section of the tubular leg openings 130. As best seen in FIG. 6B, the fixation bolt is desirably cannulated 640 to allow the bolt to follow and slid over a guide wire placed through a path drilled through the glenoid.

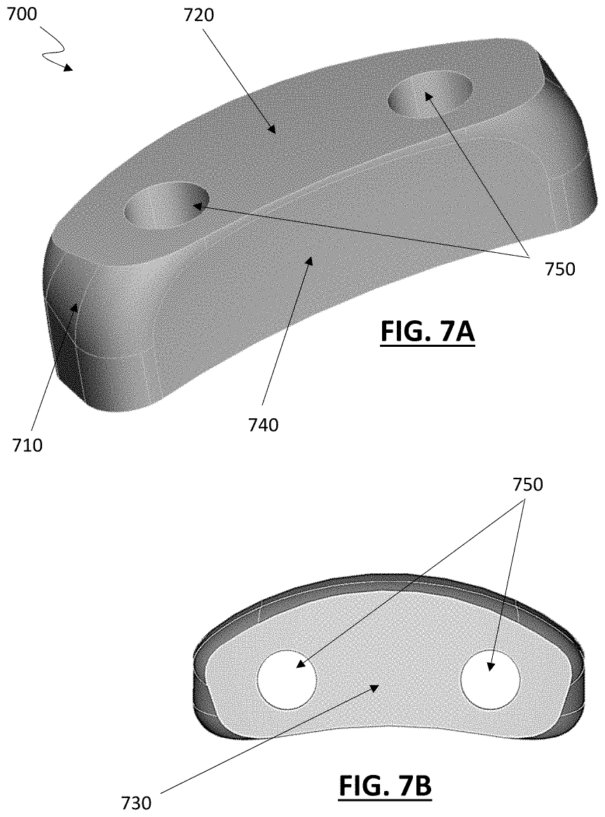

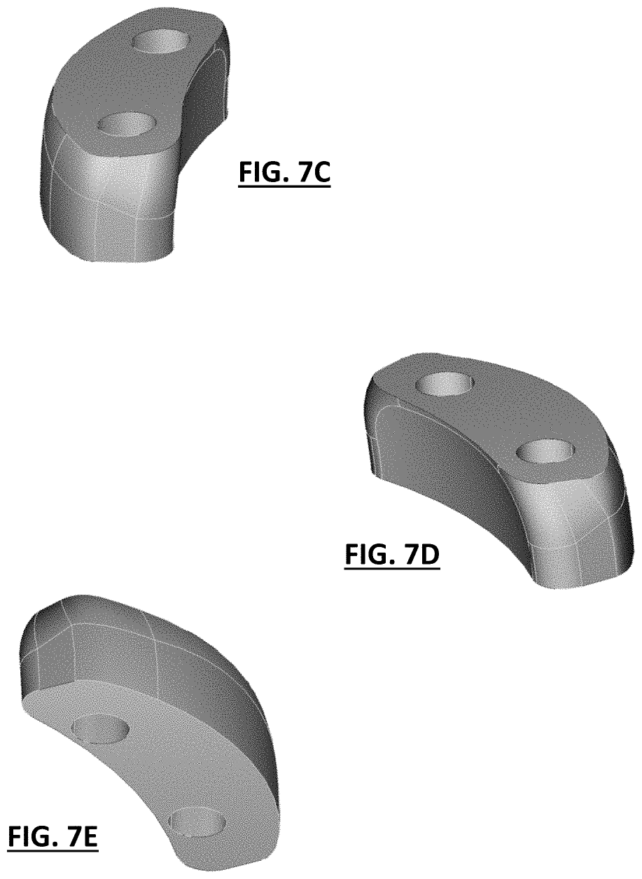

FIGS. 7A through 7E depict various views of one exemplary embodiment of a graft 700 that can be utilized with the glenoid plate of FIG. 3. In this embodiment, the graft 700 may comprise a central body 710 formed from an allograft or autograft material (or in some embodiments possibly a xenograft material), or the graft may alternatively comprise an artificial material such as a metal, plaster or ceramic body. Desirably, the graft will include an upper surface 720, a lower surface 730, an articulating surface 740 and a pair of tubular openings 750 that can be drilled or otherwise formed through the body of the graft 700 to accommodate the legs 120 of the glenoid plate 100 inside of the graft. Desirably, the graft 700 can have a curvature substantially similar to the curvature of the glenoid plate, which will also desirably emulate the natural curvature of the glenoid portion that the graft replaces. In various embodiments, the graft may be shaped from an allograft or autograft tissue by a surgeon or technician using a rongeur, mill or similar device (either before or during the surgical procedure), or a surgical graft collection tool can be utilized which cuts a suitably shaped and/or sized graft from donor material (including material from the patient's own donor site), if desired.

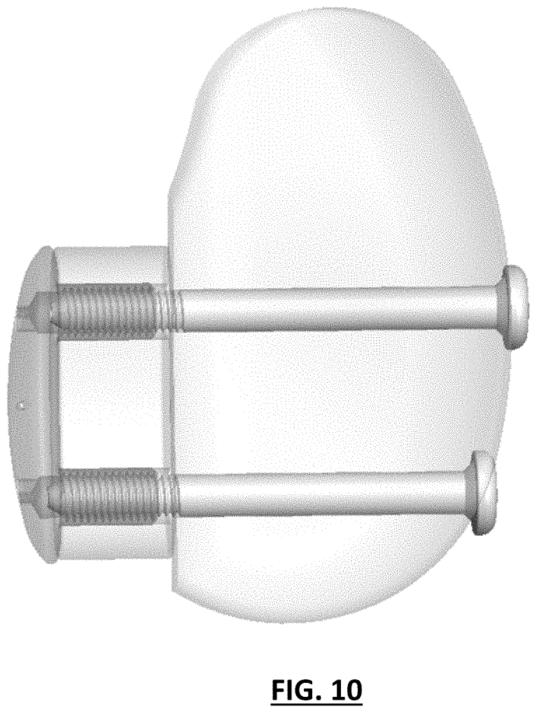

FIG. 8A depicts an exemplary placement of a graft 700 and glenoid plate 100 of FIG. 3 on a glenoid 800 of a patient. In this embodiment, the graft 700 may comprise an allograft or autograft material, or may comprise an artificial material such as a metal, plaster or ceramic body. Desirably, a pair of openings will be drilled or otherwise formed through the body of the graft 790 to accommodate the legs 120 of the glenoid plate 100 inside of the graft. A surgeon can desirably prepare the glenoid by creating a grafting surface 820 and placing a pair of guide pins 840 through the glenoid body, the guide pins extending from a posterior surface of the glenoid, traversing through the glenoid body and exit the grafting surface 820. The guide pins can then be guided through the cannulated tubular leg openings and are threaded into the upper openings 140, as best shown in FIG. 8B. A cannulated drill can then be utilized to create a pair of channels beneath the glenoid surface along the guide pins, with the drill removed and a fixation bolt advanced down each guide pin, through the channels and into the cannulated tubular leg openings. The fixation bolts can be rotated and secured into the legs, which desirably draws the graft into intimate and secure contact with the grafting surface of the glenoid. The guide pins can then be removed (see FIG. 10) and the surgery completed as necessary.

As best seen in FIG. 10, the disclosed embodiment will provide an extremely secure and durable fixation of the graft material to the glenoid. The positioning of the wide proximal heads of the fixation bolts against the posterior surface of the glenoid (i.e., against the cortical bone surface) and the compression of the glenoid between the posterior surfaces and the prepared graft surface significant reinforces and rigidifies the graft material in its desired implantation position, wherein the graft itself is even further internally supported by the legs and underside of the glenoid plate attached thereto. These arrangements provide a truly durable glenoid repair which can facilitate faster healing and return to function of the patient with little to no discomfort.

While FIG. 9 depicts the legs of the glenoid plate contained fully inside of the graft material, it should be understood that in alternative embodiments, the legs of the glenoid plate could be selected such that one or both of these legs extended to or beyond the lower surface of the graft, including embodiments wherein one or both of the legs of the glenoid plate extend fully through the graft and further extend into the tunnels formed into and/or through the glenoid itself. Such leg extension could be only a few millimeters into the glenoid up to and including extending fully through the glenoid.

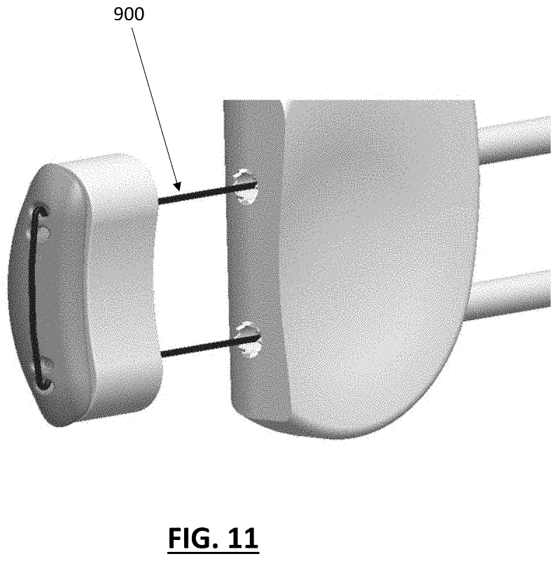

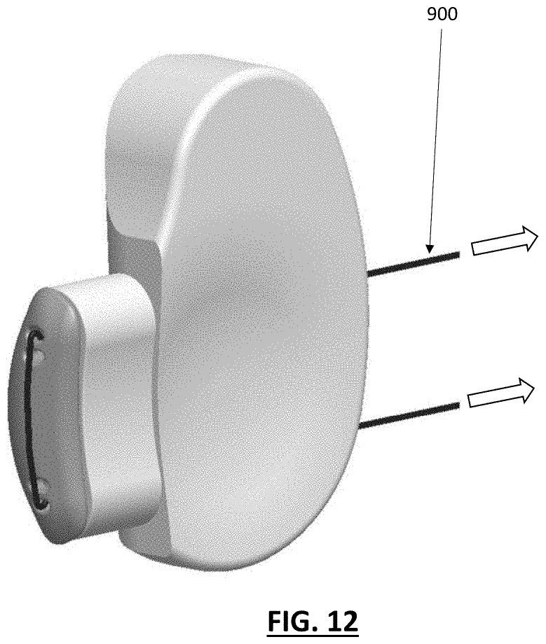

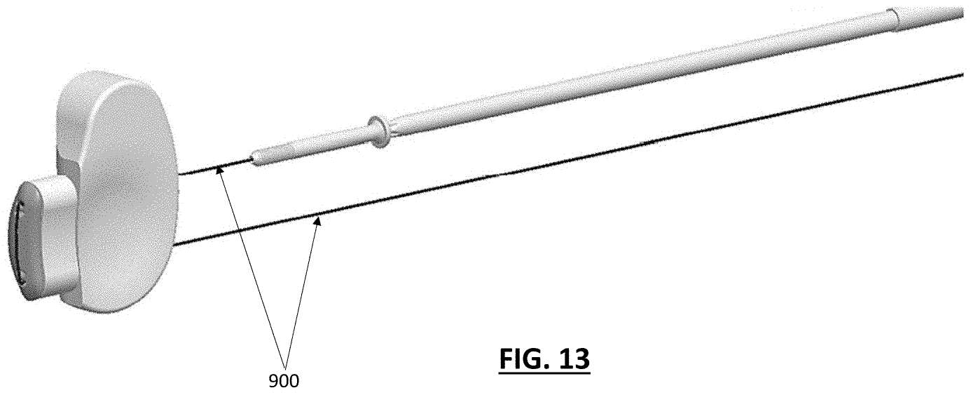

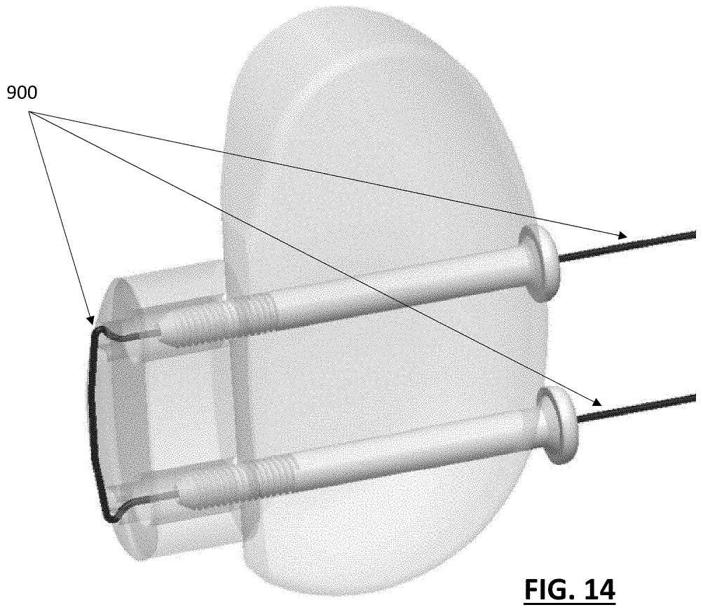

FIG. 11 depicts one alterative placement technique for securing a graft and glenoid plate to a glenoid body, in which a suture 900 is passed through the slots of the glenoid plate, with the suture extending down through and exiting out of the cannulated tubular leg openings, and extending through the channels formed in the glenoid body. In this embodiment, the suture can be drawn through the openings to pull the graft and glenoid plate into intimate contact with the grafting surface of the glenoid body (see FIG. 12), and the sutures may be advanced through the cannulated fixation bolts and a cannulated driving tool, if desired (see FIG. 13). Once the fixation bolts are secured and tightened (see FIG. 14), the suture 900 may be removed and the surgery completed.

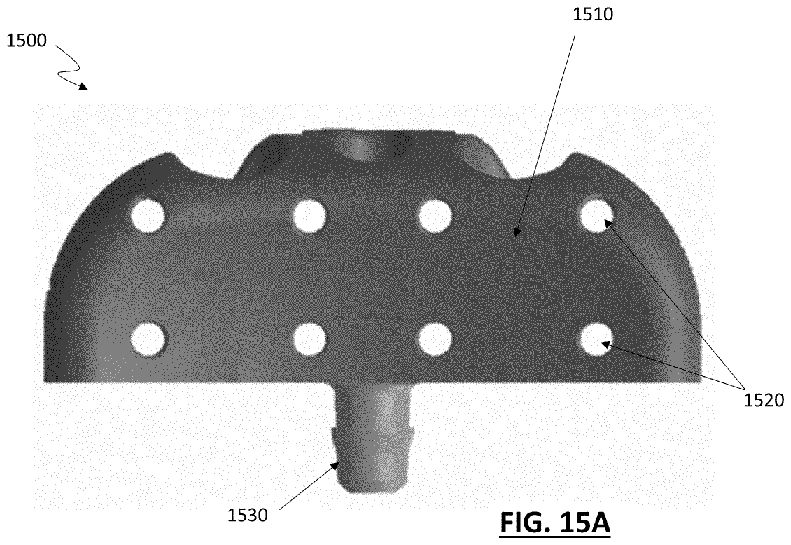

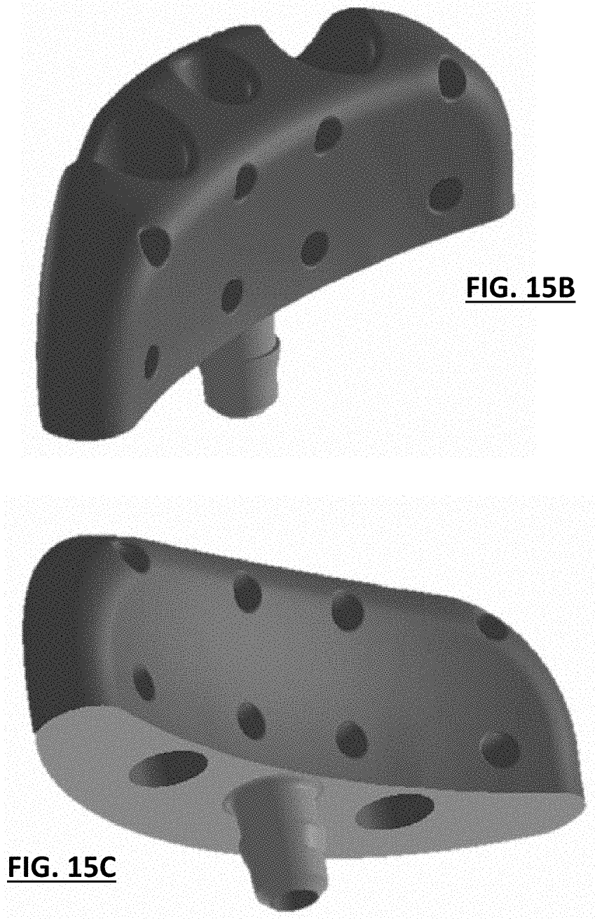

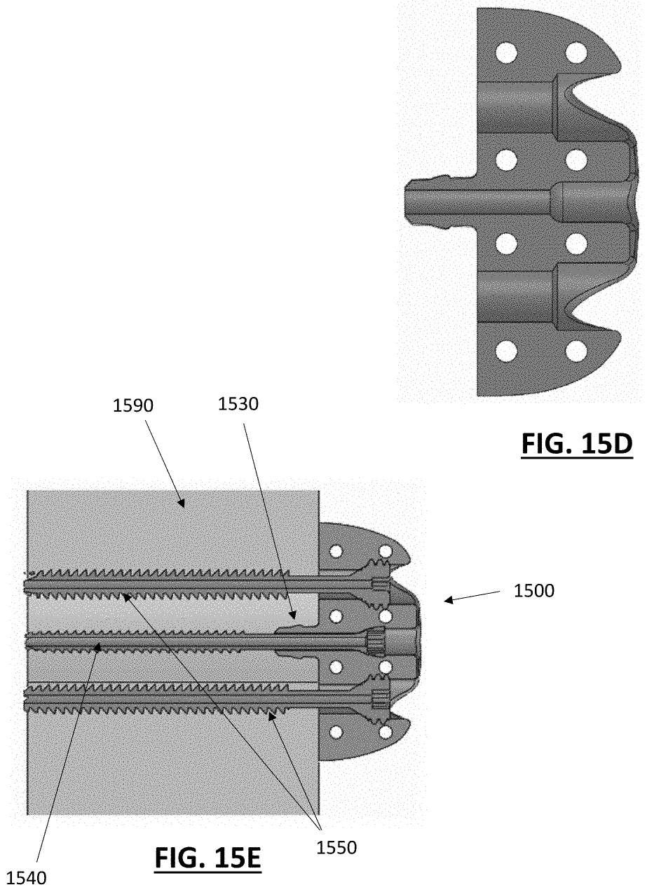

FIGS. 15A through 15D depict various views of another exemplary embodiment of a glenoid plate 1500, in which the plate comprises a titanium (or other artificial material) body 1510 which can optionally be coated with hydroxyapatite or similar known biocompatible coating materials. The body 1510 can include a curved geometry which desirably replicates the natural curved geometry of the glenoid. In various embodiments, the body 1510 can include a plurality of holes or openings 1520 which can be utilized as securement points for soft tissue repair. A cannulated peg 1530 extends from a bottom surface of the body 1510, which desirably aids in fixation and compression to the glenoid surface. Desirably, the glenoid plate 1500 can be provided in a kit with differing sizes and/or shapes of implants to be selected by the physician. FIG. 15D depicts a cross sectional view of the glenoid plate.

FIG. 15E depicts a cross sectional view of the glenoid plate 1500 of FIG. 15A when secured to a glenoid 1590. In this embodiment, a compression screw 1540 extends through the cannulate peg 1530 into the glenoid, and a pair of locking screws 1550 are secured into the glenoid on each side of the compression screw. If desired, the screws can extend outward from the plate as depicted in the figure, or the screws could alternatively extend inward to the plate (in a manner similar to the embodiments of FIGS. 2A and 2B).

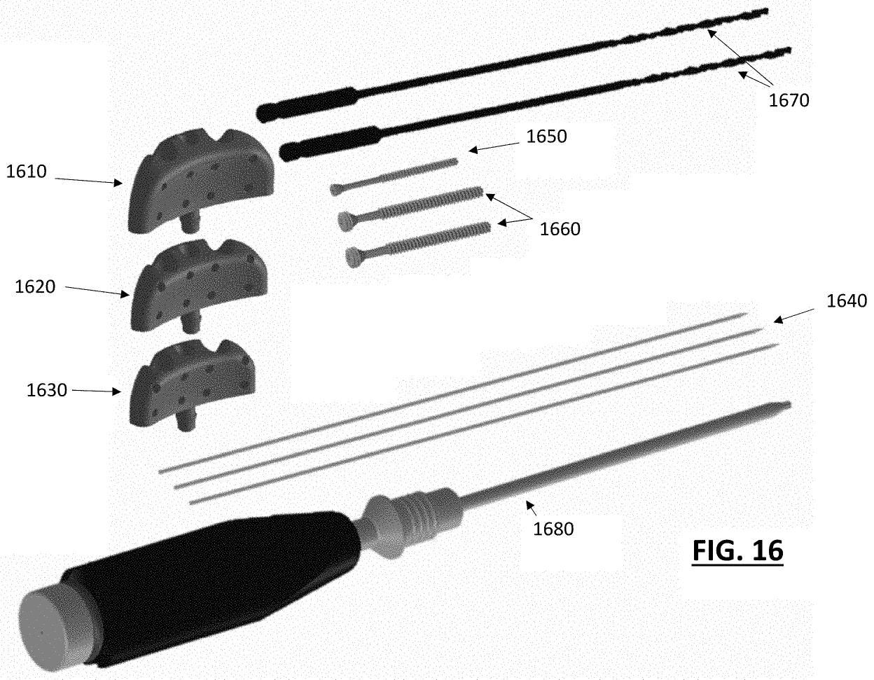

FIG. 16 depicts one exemplary surgical kit for partial glenoid repair, including a plurality of glenoid plates 1610, 1620 and 1630 of differing sizes and/or shapes, a plurality of guide pins 1640, a compression screw 1650, and a pair of locking screws 1660 a pair of cannulated drills 1670 and a surgical driving tool 1680.

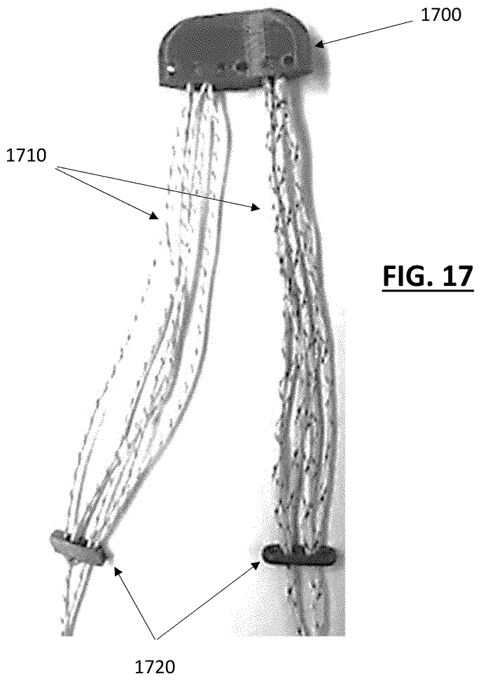

FIG. 17 depicts another alternative embodiment of a glenoid plate 1700, in which the plate 1700 can be secured to the glenoid using a plurality of securement threads or sutures 1710. In this embodiment, the distal suture ends can be secured to the glenoid using suture buttons 1720 to draw tension on the sutures and compress the glenoid plate 1700 to the glenoid in a manner similar to the other embodiments discussed herein.

While various of the disclosed embodiments are described in connection with the repair and/or replacement of an anterior rim of a glenoid, it should be understood that the disclosed devices and methods could be modified to address deficiencies in other regions of the glenoid, including other rim regions (including anterior, posterior, superior, inferior and/or other combination thereof) as well as other portions of the glenoid. Similarly, while the treatment of a diseased or damaged glenoid is disclosed and/or described herein, it should be understood that the disclosed devices and/or methods may be modified and/or may be equally useful for the treatment or other anatomical regions, including the hip socket and/or other joint structures of the body.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive, and it is to be understood that all the terms used herein are descriptive rather than limiting, and that many changes, modifications, and substitutions may be made by one having ordinary skill in the art without departing from the spirit and scope of the invention.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or dearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A glenoid implant for a shoulder prosthesis for implantation in the glenoid of a scapula, the glenoid implant comprising:
a central body formed from an artificial material having a plurality of downwardly extending legs, each of the downwardly extending legs including a canulated, internally threaded region, and
a tissue graft, the tissue graft having a plurality of openings formed therein, the plurality of openings sized and configured to fit over the plurality of downwardly extending legs when the tissue graft is assembled to the central body, the tissue graft further including an articulation surface on an external surface of the tissue graft;
a plurality of fixation bolts, each of the plurality of fixation bolts including an enlarged proximal head and a distally threaded portion, the distally threaded portion sized and configured to rotatably engage with the internally threaded region of at least one of the plurality of downwardly extending legs,
wherein, when the central body, tissue graft and plurality of fixation bolts are attached to the glenoid of the scapula, the central body compresses the tissue graft into intimate contact with the glenoid such that the articulation surface of the tissue graft is positioned proximate to a natural articulating surface of the glenoid.

2. The glenoid implant of claim 1, wherein the fixation bolts are positioned within a plurality of tunnels formed completely through the glenoid.

3. The glenoid implant of claim 1, wherein the downwardly extending legs are contained fully within the tissue graft when the tissue graft is assembled to the central body.

4. The glenoid implant of claim 1, wherein the downwardly extending legs extend fully through and outside of the tissue graft when the tissue graft is assembled to the central body.

5. The glenoid implant of claim 1, wherein the tissue graft comprises an allograft tissue graft.

6. The glenoid implant of claim 1, wherein the tissue graft comprises an autograft tissue graft.

7. The glenoid implant of claim 1, wherein the tissue graft comprises a xenograft tissue graft.

8. The glenoid implant of claim 1, wherein the tissue graft comprises an artificial tissue graft.

9. The glenoid implant of claim 9, wherein the artificial tissue graft is coated with hydroxyapatite.

10. A method of treating a patient, comprising the steps of:
identifying a patient having an injured or diseased glenoid rim surface;
removing at least a portion of the injured or diseased glenoid rim surface to create a graft receiving surface;
drilling a plurality of cavities through the glenoid, each cavity extending from a rim surface of the glenoid through the graft receiving surface;
attaching a graft material to a fixation plate, the fixation plate having a plurality of legs which extend at least partially through the graft material;
placing at least one of a plurality of fixation bolts into each of the plurality of cavities, an enlarged head portion of each fixation bolt positioned adjacent to the posterior rim surface of the glenoid and a distal end of each fixation bolt engaged with at least one of the plurality of legs of the fixation plate;
wherein the fixation plate and the plurality of fixation bolts compress the graft material against the graft receiving surface such that at least a portion of the graft material replaces the removed portion of the injured or diseased glenoid rim surface.

11. The method of claim 10, wherein the portion of the graft material which replaces the removed portion of the injured or diseased glenoid rim surface comprises an articulating surface.

12. The method of claim 10, wherein the portion of the injured or diseased glenoid rim is an anterior portion of the glenoid rim.

13. The method of claim 10, wherein the portion of the injured or diseased glenoid rim is a posterior portion of the glenoid rim.

14. The method of claim 10, wherein the portion of the injured or diseased glenoid rim is a superior portion of the glenoid rim.

15. The method of claim 10, wherein legs are recessed within the graft material.

16. The method of claim 10, wherein legs extend outward from the graft material.

17. The method of claim 10, wherein legs are flush with an external surface of the graft material.

18. The method of claim 10, further comprising the step of removing the graft material from a donor site of the patient.

19. The method of claim 10, wherein each of the plurality of fixation bolts is cannulated along a longitudinal axis.

20. The method of claim 10, wherein the plurality of cavities are recessed below an articulation surface of the glenoid.
